(19) United States
(12) Patent Application Publication
(10) Pub. No.: US 2020/0281673 A1
(43) Pub. Date: Sep. 10, 2020

(54) ACTUATOR DEVICE, END EFFECTOR, AND SURGICAL SYSTEM

(71) Applicant: SONY CORPORATION, TOKYO (JP)

(72) Inventors: HIROYUKI SUZUKI, TOKYO (JP); KAZUHITO WAKANA, KANAGAWA (JP)

(21) Appl. No.: 16/644,522

(22) PCT Filed: Aug. 1, 2018
(86) PCT No.: PCT/JP2018/028947

§ 371 Date: Mar. 5, 2020

(30) Foreign Application Priority Data
Sep. 14, 2017 (JP) 2017-176636

(51) Int. Cl. A61B 34/37; A61B 17/29; H01F 7/06

(52) U.S. Cl. CPC A61B 34/37; A61B 2562/0266; H01F 7/06; A61B 17/29

(57) ABSTRACT

Provided are an actuator device applied to a surgical system, and the like. The actuator device includes: a first magnetic body portion; a first system movable in a predetermined direction or an opposite direction of the predetermined direction; a second system including a second magnetic body portion that moves the first system in the predetermined direction by magnetic force generated between the second magnetic body portion and the first magnetic body portion, and a pressurizing portion capable of applying, to the first system, force in an opposite direction of the predetermined direction and including an elastic body and the like; and a driving unit capable of applying, to the second system, force in the predetermined direction or the opposite direction by driving. The first system includes a supporting portion configured to support an acting portion that acts by a reciprocating motion in the predetermined direction, and the second system includes a sliding portion connected to the supporting portion via the elastic portion.

### TECHNICAL FIELD

The technology disclosed in the present specification relates to: an actuator device applied to, for example, a surgical system; an end effector of the surgical system; and the surgical system.

### BACKGROUND ART

Recently, a robotics technology has made remarkable progress, and the robotics technology has widely spread in workplaces of various industrial fields. A master-slave robot system is used in industrial fields where it is still difficult to perform full autonomous operation under the control of a computer, such as a medical robot. For example, a surgeon uses a master-slave medical robot for endoscopic surgery for an abdominal cavity, a chest cavity, or the like, and can carry out the surgery by remotely operating a slave arm, to which a surgical tool such as a forceps is attached, while viewing an operative field on a 3D monitor screen.

Considering invasion to living tissue and efficiency of surgical treatment under the endoscope, it is preferable that external force received by the end effector of the slave from an affected site and the like is presented to a user on the master side. As of this master-slave robot system, several proposals have been made for a medical robot capable of detecting force acting on an end effector such as a gripping portion (gripper). Furthermore, a proposal also has been made for a medical instrument and a medical support arm device capable of detecting contact force (see Patent Document 1, for example).

In a surgical robot utilized in an endoscopic surgery, downsizing a configuration of an end effector is essential. Therefore, it is general to employ a driving mechanism in which driving force generated in a driving unit like an actuator or the like arranged apart from the end effector is transmitted by a wire or cable to open/close the end effector. However, in a configuration in which a force sensor is disposed between the end effector and the driving unit that drives the end effector, traction force of the wire to open/close the end effector interferes with, for example, external force applied in a long axis direction of the end effector, and therefore, there are concerns that sensitivity of the force sensor may be degraded or calibration may become difficult.

On the other hand, there is a known pair of surgical forceps including: a pair of jaw members respectively having cam slots bored and coupled to each other in an openable/closable manner; and a shaft having an elongated shape and including a cam pin that is positioned at a tip and inserted into the cam slots, in which the pair of surgical forceps opens/closes the jaw members by reciprocating the elongated shaft in a longitudinal direction to make the cam pin slide inside the cam slots (see Patent Document 2, for example). In this type of forceps, when gripping force is increased, frictional force between the cam slots is increased, and traction force via the shaft is largely lost before being transmitted as the gripping force by the jaw members. To obtain desired large gripping force, it is necessary to increase the traction force by an amount compensating for the frictional force. Therefore, there is a problem that output of an actuator that generates the traction force is required to be increased.

### CITATION LIST

### Patent Document

Patent Document 1: Japanese Patent Application Laid-Open No. 2017-29214
Patent Document 2: Japanese Patent Application Laid-Open No. 2008-188440

### SUMMARY OF THE INVENTION

### Problems to be Solved by the Invention

An object of the technology disclosed in the present specification is to provide: an actuator device applied to a surgical system; an end effector of the surgical system; and the surgical system.

### Solutions to Problems

The technology disclosed in the present specification is made in consideration of the problems described above, and according to a first aspect thereof, provided is an actuator device including:

a first magnetic body portion;

a first system movable in a predetermined direction or an opposite direction of the predetermined direction;

a second system including a second magnetic body portion that moves the first system in the predetermined direction by magnetic force generated between the second magnetic body portion and the first magnetic body portion, and a pressurizing portion capable of applying, to the first system, force in the opposite direction of the predetermined direction and including an elastic body and the like; and

a driving unit capable of applying, to the second system, force in the predetermined direction or the opposite direction by driving. The more the first system is drawn in the predetermined direction, the more the force in the opposite direction of the elastic portion is increased. Furthermore, the first system includes a supporting portion configured to support an acting portion that acts by a reciprocating motion in the predetermined direction.

The second system includes a sliding portion connected to the supporting portion via the elastic portion. The sliding portion has one surface that is oriented in a direction parallel to the predetermined direction and connected to the elastic portion, has the other surface connected to the second magnetic body portion, and is relatively movable in the direction parallel to the predetermined direction by the driving of the driving unit.

The supporting portion has a hollow structure. Additionally, the sliding portion is housed inside the hollow structure and relatively movable in a direction parallel to the predetermined direction.

Furthermore, a second magnetic body portion attached to the sliding portion in a manner facing the magnetic body portion is further provided, and the magnetic body portion sucks the second magnetic body portion by magnetic force.

Additionally, the driving unit includes, for example, a dielectric elastomer and is driven in the predetermined direction by extension/contraction.

In a state where the first system is positioned closest to the magnetic body portion, attraction force by magnetic force of the first magnetic body portion and the magnetic force of the second magnetic body portion is larger than restoring force of the elastic portion. Furthermore, in a case where the second system separates the first system from the first magnetic body portion, the driving unit generates driving force in the opposite direction of the predetermined direction, the driving force being larger than a difference between the attraction force by the magnetic force of the first magnetic body portion and the restoring force of the elastic portion.

Furthermore, according to a second aspect of the technology disclosed in the present specification, provided is an end effector including:

a gripping portion; and an actuator unit that generates traction force to the gripping portion, in which

the actuator unit includes

a first magnetic body portion,

a first system movable in a predetermined direction or an opposite direction of the predetermined direction,

a second system including a second magnetic body portion that moves the first system in the predetermined direction by magnetic force generated between the second magnetic body portion and the first magnetic body portion, and a pressurizing portion capable of applying, to the first system, force in the opposite direction of the predetermined direction, and

a driving unit capable of applying, to the second system, force in the predetermined direction or the opposite direction by driving.

Moreover, according to a third aspect of the technology disclosed in the present specification, provided is a surgical system including:

an end effector;

an actuator unit that generates traction force to the end effector; and

a force sensor arranged closer to a proximal end side than the actuator unit.

The force sensor includes, for example, a strain detection element that detects strain of a strain element and includes an FBG sensor.

Furthermore, according to a fourth aspect of the technology disclosed in the present specification, provided is a surgical system including:

an end effector; and an actuator unit that generates traction force to the end effector, in which

the actuator unit includes

a first system that is sucked by magnetic force of a magnetic body portion and moves, in a predetermined direction, an acting portion that causes the traction force to act on the gripping portion, and

a second system that applies, to the first system, force in an opposite direction of the predetermined direction, and separates the first system from the magnetic body portion.

### Effects of the Invention

According to the technology disclosed in the present specification, it is possible to provide the actuator device applied to the surgical system, the end effector of the surgical system, and the surgical system.

Note that the effect described in the present specification is an example, and the effect of the present invention is not limited thereto. Furthermore, there may be a case where the present invention further provides an additional effect other than the above-described effect.

Other objects, features, and advantages of the technology disclosed in the present specification will be further described in more detail on the basis of embodiments as described later and the attached drawings.

### BRIEF DESCRIPTION OF DRAWINGS

FIG. 1 is a view illustrating an exemplary configuration of a surgical robot 100 to which a technology disclosed in the present specification is applied.

FIG. 2 is a view illustrating a modified example of the surgical robot 100.

FIG. 3 is a view illustrating an exemplary configuration of an actuator unit 102.

FIG. 4 is a view illustrating the exemplary configuration of the actuator unit 102.

FIG. 5 is a view illustrating force acting on a first system.

FIG. 6 is a diagram illustrating force acting on a second system.

FIG. 7 is a diagram illustrating exemplary calculation of generative force in accordance with a displacement amount of the actuator unit 102.

FIG. 8 is a diagram illustrating exemplary calculation of gripping force of a gripping portion 101 in accordance with the displacement amount of the actuator unit 102.

FIG. 9 is a diagram illustrating exemplary calculation of the generative force in accordance with the displacement amount of the actuator unit 102.

FIG. 10 is a view illustrating an exemplary configuration of a force sensor 103.

FIG. 11 is a view illustrating an XY cross section at a position a of a strain element 1001.

FIG. 12 is a view to describe a mechanism of detecting force acting on the strain element 1001.

FIG. 13 is a diagram to describe a method of installing, on the strain element 1001, a strain detection element utilizing an FBG sensor.

FIG. 14 is a diagram illustrating a processing algorithm of a 4DOF force sensor.

FIG. 15 is a view illustrating exemplary implementation of the actuator unit 102.

FIG. 16 is a view illustrating a first system of the actuator unit 102.

FIG. 17 is a view illustrating a second system of the actuator unit 102.

FIG. 18 is a view illustrating exemplary operation of the actuator unit 102.

FIG. 19 is a view illustrating exemplary operation of the actuator unit 102.

FIG. 20 is a view illustrating exemplary operation of the actuator unit 102.

FIG. 21 is a view illustrating exemplary operation of the actuator unit 102.

FIG. 22 is a view illustrating exemplary operation of the actuator unit 102.

FIG. 23 is a view illustrating exemplary operation of the actuator unit 102.

FIG. 24 is a view illustrating exemplary operation of the actuator unit 102.

FIG. 25 is a view illustrating exemplary operation of the actuator unit 102.

### MODE FOR CARRYING OUT THE INVENTION

Hereinafter, an embodiment of a technology disclosed in the present specification will be described in detail with reference to the drawings.

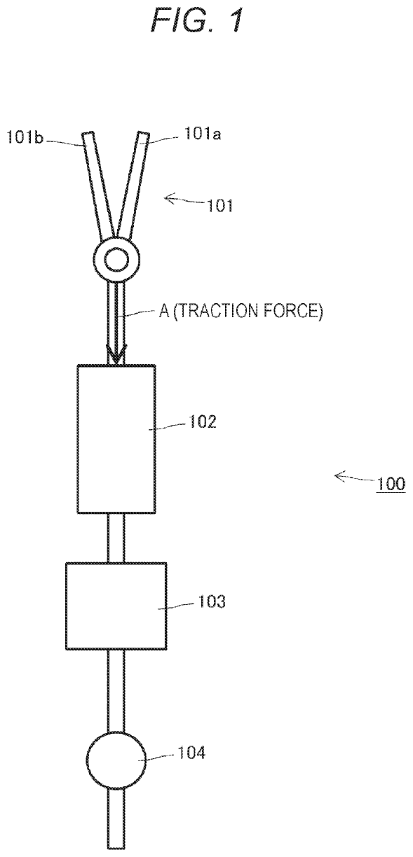

FIG. 1 schematically illustrates an exemplary configuration of a surgical robot 100 to which the technology disclosed in the present specification is applied. The illustrated surgical robot 100 includes, for example, an arm robot and is provided with, sequentially from a distal end side more than a bend portion 104 such as a joint: a gripping portion 101 as an end effector; an actuator unit 102 that supplies gripping traction force to the gripping portion 101; and a force sensor 103 to detect external force acting on the gripping portion 101.

The gripping portion 101 is a pair of surgical forceps and includes a pair of blades 101a and 101b coupled in an openable/closable manner. When the blades 101a and 101b are opened/closed by being driven in directions opposing to each other, and can grip living tissue. A coupled portion between the respective blades 101a and 101b has a mechanical structure that converts traction force in a linear movement direction into gripping force. Therefore, when the traction force in the linear movement direction acts on the gripping portion 101 as indicated by an arrow A in the drawing, the blades 101a and 101b are closed, and when force in an opposite direction of the arrow A acts on the gripping portion 101, the blades 101a and 101b are opened.

For example, cam slots are bored on the respective blades 101a and 101b, a cam pin protruding at a tip portion of an elongated shaft is inserted into the cam slots, and the pair of blades can be opened/closed by reciprocating the elongated long shaft in a longitudinal direction to make the cam pin slide inside the cam slots (see Patent Document 2, for example). Note that the structures of the cam and the slots are not illustrated to simplify the drawing.

The actuator unit 102 includes, for example, an acting portion that performs linear movement, and can supply traction force, through the acting portion, for reciprocating the elongate-shaped shaft of the gripping portion 101 as the pair of surgical forceps.

For example, large gripping force, such as gripping a needle with strong force during surgery, is necessary when an open/close angle of the gripping portion 101 becomes close to zero degrees. In the present embodiment, the actuator unit 102 generates the large traction force when the open/close angle of the gripping portion 101 becomes close to zero degrees. Note, however, that a detailed configuration of the actuator unit 102 will be described later.

The force sensor 103 includes, for example, a six-axis force sensor and can detect: triaxial force acting on the gripping portion 101 provided as the end effector; and torque around the respective axes. A detailed configuration of the force sensor 103 will be described later.

The surgical robot 100 according to the present embodiment has the gripping portion 101, the actuator unit 102, and the force sensor 103 which are sequentially disposed from the distal end side toward a proximal end. In other words, the force sensor 103 is arranged in a region located between the actuator unit 102 and the proximal end and free from acting of the traction force to generate the gripping force of the gripping portion 101. According to such a configuration, the traction force by the actuator unit 102 does not reach the force sensor 103. Since the traction force of the actuator unit 102 does not interfere with the external force applied in a long axis direction of the end effector, sensitivity of the force sensor 103 is not degraded and a detection signal from the force sensor 103 can be easily calibrated.

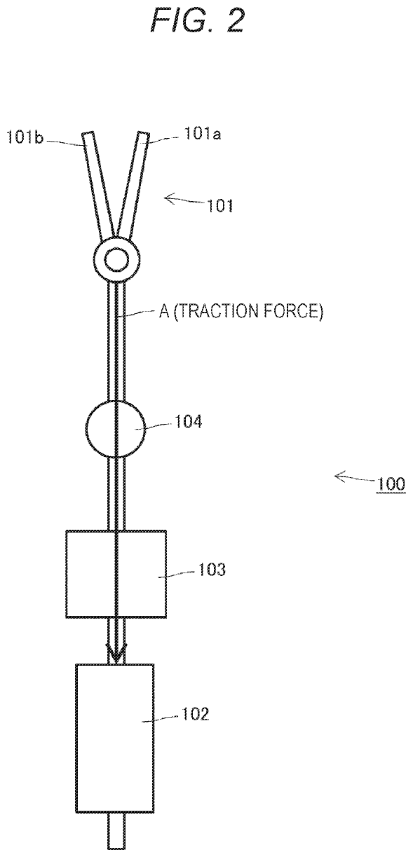

FIG. 2 illustrates a modified example of the surgical robot 100 for comparison with FIG. 1. In the surgical robot 100 according to the illustrated modified example, the gripping portion 101, a bend portion 104, the force sensor 103, and the actuator unit 102 are sequentially disposed from a distal end side. Note, however, that constituent elements same as those illustrated in FIG. 1 are denoted by the same reference signs.

Main differences from the exemplary configuration illustrated in FIG. 1 are that: the bend portion 104 is interposed between a portion including the force sensor 103 and the actuator unit 102 and the gripping portion 101 and; and the force sensor 103 is disposed on the distal end side (or close to the gripping portion 101) more than the actuator unit 102. In the configuration in which the force sensor 103 is arranged between the gripping portion 101 and the actuator unit 102, the traction force by the actuator unit 102 reaches the force sensor 103. In other words, the traction force of the actuator unit 102 interferes with the external force applied in the long axis direction of the end effector. Due to this, there is a problem that the sensitivity of the force sensor 103 is degraded and calibration of the force sensor 103 becomes difficult.

As described above, according to the configuration of the surgical robot 100 illustrated in FIG. 1, the sensitivity of the force sensor 103 can be improved. On the other hand, in a case where the actuator unit 102 is arranged in the vicinity of the distal end, downsizing is required, and therefore, there is a problem that output of an actuator is reduced. For example, large gripping force, such as gripping a needle with strong force during surgery, is necessary when the open/close angle of the gripping portion 101 becomes close to zero degrees. Considering this, the present specification proposes a structure of the actuator unit 102 that can be downsized and is capable of extracting the large gripping force even with little driving force.

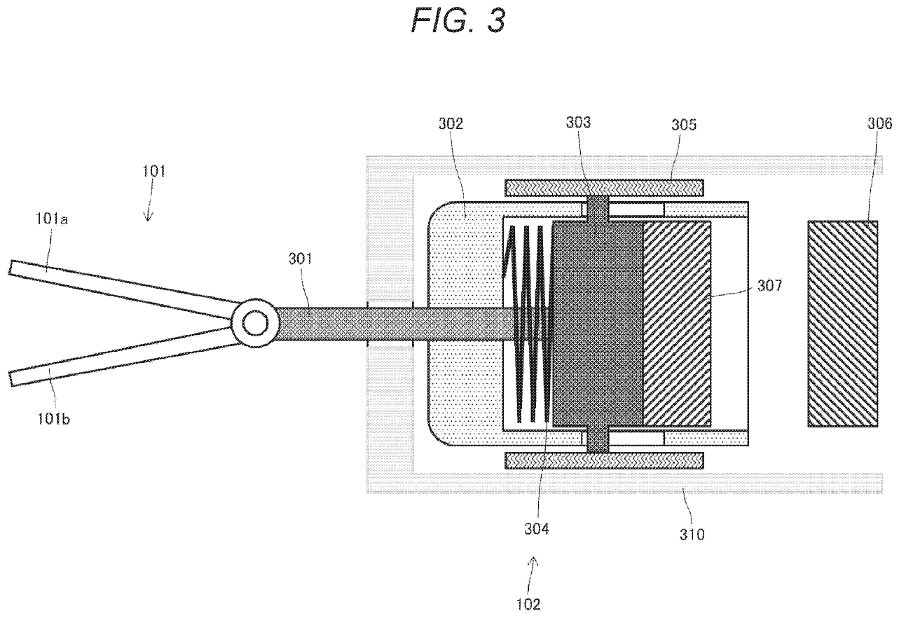

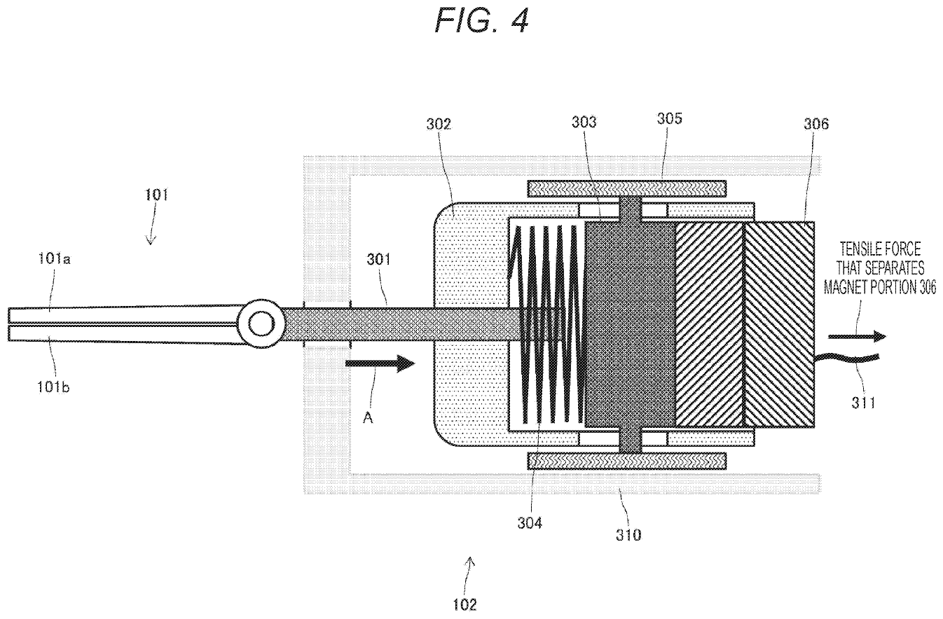

FIGS. 3 and 4 illustrate an exemplary configuration of the actuator unit 102 proposed in the present specification. Both FIGS. 3 and 4 illustrate a cross section of the actuator unit 102. Note, however, that FIG. 3 illustrates a state where the traction force to generate the gripping force of the gripping portion 101 is not acting (that is, corresponding to a state where the gripping portion 101 is opened), and FIG. 4 illustrates a state where the traction force is acting (that is, corresponding to a state where the gripping portion 101 is closed).

The actuator unit 102 generates traction force in a linear movement direction indicated by the arrow A in FIG. 3, and includes: an acting portion 301 that causes the traction force to act on the gripping portion 101; a supporting portion 302 supporting the acting portion 301; and a sliding portion 303 relatively movable in a direction parallel to the arrow A with respect to the supporting portion 302.

The supporting portion 302 has a hollow cylindrical shape, and an axis of the cylinder is parallel to the arrow A. Furthermore, the sliding portion 303 is housed inside the cylinder and can be relatively moved in the direction parallel to the arrow A with respect to the supporting portion 302 by the sliding portion sliding or slipping along an inner wall of the cylinder. Therefore, a portion including the acting portion 301 and the supporting portion 302 and the sliding portion 303 are basically constrained so as to be relatively moved only in the direction parallel to the arrow A. The sliding portion 303 can also be referred to as an internal component of the supporting portion 302.

The sliding portion 303 has one end surface that is oriented in the arrow A direction and connected to a bottom surface portion of the hollow cylinder on the supporting portion 302 side via an elastic portion 304 including a coil spring or the like. Therefore, when a relative position between the supporting portion 302 and the sliding portion 303 is changed in the linear movement direction indicated by the arrow A or in an opposite direction thereof, restoring force Fₖ of the elastic portion 304 acts in a direction returning to an original position. The coil spring used for the elastic portion 304 has, for example, a linear characteristic, and the restoring force Fₖ thereof is directly proportional to a displacement amount Δx from a natural length of the coil spring. Using a spring constant k, it can be expressed as Fₖ=k·Δx. Note, however, that a non-linear spring can also be used as the elastic portion 304. Furthermore, as far as the force in an opposite direction of a predetermined direction indicated by the arrow A can be applied, the elastic portion 304 is not limited to the one including an elastic member, and a pressurizing portion can also be used as the elastic portion 304. For example, a magnet that generates attraction force in the opposite direction can also be applied as the elastic portion 304.

Furthermore, a magnetic body portion 306 that includes a permanent magnet or the like and generates magnetic force is disposed at a rear end (proximal end side) of the actuator unit 302. Additionally, the sliding portion 303 has the other end surface to which a second magnetic body portion 307 is attached in a manner facing the magnetic boy portion 306. Since the magnetic body portion 306 and the second magnetic body portion 307 are disposed in a manner such that different polarities face each other, attraction force F_{M} by the magnetic force of the magnetic body portion 306 acts on the sliding portion 303 in the predetermined direction indicated by the arrow A. Therefore, the force F_{M} in the arrow A direction is applied to the supporting portion 302 via the sliding portion 303 and the elastic portion 304, and becomes the traction force in the linear movement direction of the acting portion 301.

The attraction force F_{M} is inversely proportional to the square of a distance between the magnetic body portion 306 and the second magnetic body portion 307. Due to this, when the magnetic body portion 306 and the second magnetic body portion 307 are closest to each other and the open/close angle of the gripping portion 101 becomes close to zero degrees, the actuator unit 102 can generate large traction force by the magnetic force. Therefore, it is possible to downsize dimensions of the actuator unit 102 (particularly, in the direction orthogonal to the longitudinal direction).

Note that an electromagnet including a coil may be used instead of the permanent magnet in one or both of the magnetic body portion 306 and the second magnetic body portion 307 (note, however, that it is necessary to increase the number of turns of the coil, leading to upsize of the magnetic body portion, and also large coil current is required to generate magnetic force as much as the magnetic force of the permanent magnet. Using the permanent magnet is more inexpensive and provides a simple structure). Furthermore, even when either one of the magnetic body portion 306 and the second magnetic body portion 307 is manufactured with a magnetic body instead of a magnet, the attraction force F_{M} by the magnetic force can be made to act on a range between the sliding portion 303 and the magnetic body portion 306 (or a range between the supporting portion 302 and the magnetic body portion 306). For example, a magnetic body may constitute the entire sliding portion 303, instead of attaching the magnetic body to the other end surface of the sliding portion 303.

Furthermore, the sliding portion 303 is coupled to a driving unit 305 that is linearly moved in the direction parallel to the arrow A. Specifically, the sliding portion 303 includes protruding portions protruding to an upper end and a lower end in the drawing paper. Additionally, these protruding portions are coupled to the driving unit 305 via linear apertures bored on the cylinder portion of the supporting portion 302, and the driving unit 305 is disposed outside the supporting portion 302. The driving unit 305 is a linear movement actuator that drives the supporting portion 302 in the direction parallel to the arrow A. Therefore, driving force FA in the direction parallel to the arrow A is applied to the sliding portion 303 from the driving unit 305. As described later, the driving force FA in the opposite direction of the arrow A acts so as to pull away the sliding portion 303 from the magnetic body portion 306.

In the present embodiment, a dielectric elastomer (DEA) that is one of electro-active polymers (EAP) is used as the driving unit 305 that is the linear movement actuator. Examples of the DEA include a silicon-based polymer, a urethane-based polymer, an acrylic polymer, and the like. The DEA as the driving unit 305 is extended/contracted in the linear movement direction indicated by the arrow A, and the relative position between the portion including the acting portion 301 and the supporting portion 302 and the sliding portion 303 is changed by this configuration. Therefore, the driving force FA by the driving unit 305 includes generative force F_{DEA} by the DEA. The driving force F_{DEA} by the driving unit 305 is varied in accordance with voltage applied to the DEA. For example, the driving unit 305 includes the DEA shaped like a hollow cylinder and is disposed so as to house the supporting portion 302 inside the cylinder.

The DEA is an example of the linear movement actuator. Besides the DEA, it may be possible to use, as the driving unit 305 that is the linear movement actuator, a conductive polymer actuator, an ion conducting actuator, a macro fiber composite (MFC) actuator, a ferroelectric polymer actuator, a piezo actuator, a voice coil, a micromotor, a pneumatic cylinder, or the like. Note, however, that the present applicant considers that the DEA is preferable because of characteristics as follows: a displacement amount in the linear movement direction can be estimated from changes in dimensions, magnitude of the generative force, and a displacement amount, and changes in a displacement amount and capacitance. Note that, as for a transducer device utilizing a DEA, refer to, for example, Japanese Patent Application No. 2017-133160 already assigned to the present applicant.

Assume that main components of the actuator unit 102, such as the supporting portion 302, the sliding portion 303, the driving unit 305, and the magnetic body portion 306 described above, are accommodated in a housing 310.

The acting portion 301 and the supporting portion 302 are integrally fixed. Force that pushes the acting portion 301 in the linear movement direction indicated by the arrow A is the traction force to the gripping portion 101 coupled to the end (distal end side) of the acting portion 101. This traction force includes resultant force including: the restoring force Fₖ by the elastic portion 304; the driving force F_{DEA} by the driving unit 305; and the magnetic force F_{M} by the magnetic body portion 306. Note, however, that the restoring force Fₖ is internal force received by the supporting portion 302 from the sliding portion 303 provided as the internal component, and the restoring force is offset inside, and therefore, the restoring force does not contribute to the traction force acting on the outside. FIG. 4 illustrates the state where the traction force of the actuator unit 102 is acting. Since the acting portion 301 applies the traction force to the gripping portion 101, the gripping portion 101 is closed.

The gripping portion 101 is the pair of surgical forceps that grips living tissue, and includes the pair of blades 101a and 101b that are opened and closed by being driven in the opposing directions from each other. A coupled portion of the respective blades 101a and 101b has the mechanical structure that converts the traction force in the linear movement direction into the gripping force. Specifically, the cam slots are bored on respective blades 101a and 101b. Furthermore, the acting portion 301 includes the elongated shaft and the cam pin protrudes from the tip portion of the shaft, and the pair of blades 101a and 101b can be opened and closed by the cam pin sliding inside the cam slots. That is, when the traction force in the linear movement direction indicated by the arrow A in FIG. 3 acts on the gripping portion 101, the blades 101a and 101b are closed as illustrated in FIG. 4. Furthermore, when the force in the opposite direction of the arrow A acts on the gripping portion 101 with the blades 101a and 101b closed, the blades 101a and 101b are opened as illustrated in FIG. 3.

Note, however, that detailed configurations of the gripping portion 101 and the blades 101a and 101b are not illustrated in the drawing because: the structure of a surgical terminal that is opened/closed by converting the traction force in the linear movement direction into the gripping force is well known; and furthermore, the technology disclosed in the present specification is not limited to the structure of a specific surgical terminal.

Operation of the actuator unit 102 will be described in more detail.

The actuator unit 102 illustrated in FIGS. 3 and 4 is structurally separated into: a first system that directly influences the traction force of the gripping portion 101; and a second system that does not directly influence the traction force of the gripping portion 101. In the following, resultant force acting on the first system will be defined as F₁, and resultant force acting on the second system will be defined as F₂.

The first system includes the acting portion 301 and the supporting portion 302. Note that the sliding portion 303 is included as the internal component of the supporting portion 302, but does not belong to the first system. The first system is moved in the linear movement direction indicated by the arrow A and generates the large traction force while utilizing the magnetic force F_{M} of the magnetic body portion 306 particularly in a region where the open/close angle of the gripping portion 101 becomes close to zero degrees. Note that the restoring force Fₖ generated by the elastic portion 304 that connects the supporting portion 302 and the sliding portion 303 is the internal force received by the supporting portion 302 from the sliding portion 303 provided as the internal component, and the restoring force is offset inside, and therefore, the restoring force does not contribute to the traction force acting on the outside.

On the other hand, the second system includes the sliding portion 303, the elastic portion 304, and the second magnetic body portion 307 that is integrated with the sliding portion 303, and receives the driving force F_{DEA} from the driving unit 305 and is further applied with the restoring force Fₖ from the elastic portion 304. In the second system, if a design is made such that the restoring force Fₖ by the elastic portion 304 and the magnetic force F_{M} by the magnetic body portion 306 are cancelled each other, the second magnetic body portion 307 can be pulled away from the magnetic body portion 306 by making the second system slide in the opposite direction of the arrow A by small force F₂.

The magnetic force has a characteristic of nonlinearly attenuating relative to a distance between the magnets (specifically, the magnetic force attenuates in inverse proportion to the square of the distance between the magnets). Therefore, the actuator unit 102 obtains large gripping force on the basis of such a characteristic of the magnets by utilizing the magnetic force of the magnetic body portion 306 when the open/close angle of the gripping portion 101 is close to the zero degrees, and furthermore, the second system is made to slide even by the small driving force F_{DEA} of the driving unit 305 to open the gripping portion 101, and a gripped object can be released.

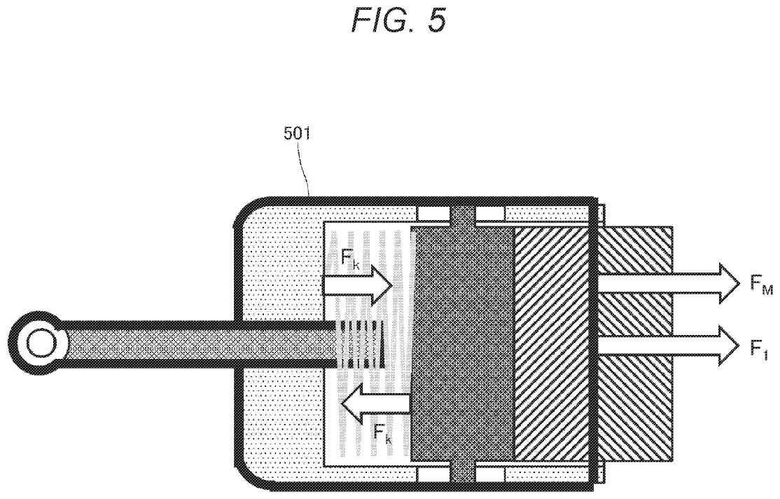

FIG. 5 illustrates the force acting on the first system when the actuator unit 102 pulls the gripping portion 101. In the drawing, the components constituting the first system are surrounded by a thick line 501. Note, however, that the sliding portion 303 included as the internal component of the supporting portion 302 is surrounded by the thick line 501, but does not belong to the first system (as described above). The resultant force F₁ of the force acting on the first system becomes the traction force to the gripping portion 101, and also becomes the gripping force when the open/close angle of the gripping portion 101 is close to the zero degrees.

The restoring force Fₖ is applied to the supporting portion 302 from the elastic portion 304. Furthermore, the attraction force F_{M} from the magnetic body portion 306 is applied to the sliding portion 303. Among these kinds of force, the restoring force Fₖ is the internal force received by the supporting portion 302 from the sliding portion 303 provided as the internal component, and the restoring force is offset inside. Therefore, in the first system, it can be said the traction force F₁ of the gripping portion 101 corresponds to the attraction force F_{M} received from the magnetic body portion 306 as represented by Expression (1) below.

[Math. 1]

F₁=F_{M}+(Fₖ−Fₖ)=F_{M} (1)

The attraction force F_{M} acts in the same direction as the traction force indicated by the arrow A, in other words, the attraction force F_{M} becomes the traction force acting on the gripping portion 101 in the linear movement direction. Therefore, when the open/close angle of the gripping portion 101 becomes close to zero degrees, the first system can generate the large traction force F₁ utilizing the magnetic force F_{M} and can lock a gripped state.

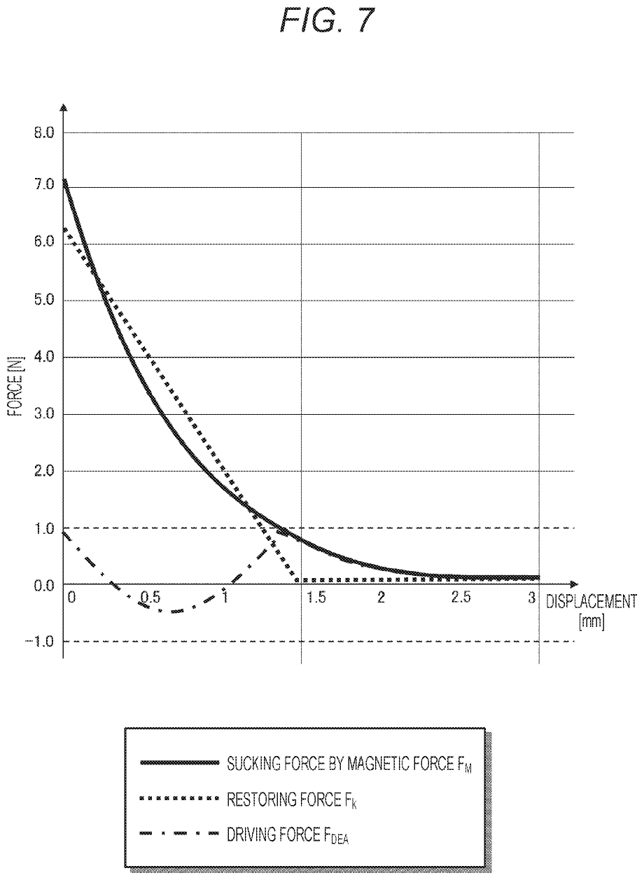

FIG. 7 illustrates exemplary calculation values of the attraction force F_{M} by the magnetic force of the magnetic body portion 306, the restoring force F_{K} of the elastic portion 304, and the generative force F_{DEA} of the driving unit (DEA) 305 when the actuator unit 102 attempts to displace the acting portion 301 in the linear movement direction indicated by the arrow A (that is, when the traction force is applied to the gripping portion 101 so as to close the gripping portion). Note that a horizontal axis represents the displacement amount of the acting portion 301 and a vertical axis represents force [N]. Furthermore, a maximum displacement amount of the actuator unit 102 is set to 3 mm, a position where the acting portion 301 is displaced maximally in the opposite direction of the arrow A is set as 0 on the horizontal axis, and the linear movement direction indicated by the arrow A is defined as a positive direction of the horizontal axis. Additionally, the calculation is made while setting an elastic coefficient of the elastic portion 304 as k=4.5 N/mm.

The attraction force F_{M} by the magnetic force of the magnetic body portion 306 is increased in inverse proportion to the distance from the second magnetic body portion 307. Furthermore, the elastic portion 304 includes the coil spring having, for example, the linear characteristic, and the restoring force F_{K} thereof is increased in proportion to the distance from where the displacement amount is close to 1.5 mm. Therefore, the more the displacement amount is increased and the smaller the open/close angle of the gripping portion 101 becomes, the more the gripping force is nonlinearly increased. Furthermore, the restoring force F_{K} of the elastic portion 304 has the linear characteristic, and a magnitude relation with the attraction force F_{M} by the magnetic force of the magnetic body portion 306 is reversed in the process in which the acting portion 301 is displaced, but an insufficient force is compensated by the generative force F_{DEA} of the driving unit 305. It is found that when the generative force F_{DEA} of the driving unit 305 is in a range of −1 to +1 [N], the actuator unit 102 is operable.

A rightmost end of the horizontal axis of the graph illustrated in FIG. 7 is the maximum displacement position of the actuator unit 102 where the magnetic body portion 306 closely contacts (or is positioned closest to) the second magnetic body portion 307. The gripping portion 101 should be designed and accurately attached to the end (distal end side) of the acting portion 301 such that the gripping portion 101 is completely closed at this maximum displacement position. Furthermore, the gripping portion 101 can be brought into a grip lock state by selecting a coil spring used for the elastic portion 304 such that the attraction force F_{M} by the magnetic force of the magnetic body portion 306 becomes larger than the restoring force F_{K} of the elastic portion 304 at the maximum displacement position of the actuator unit 102.

Note that FIG. 7 illustrates the exemplary calculation in the case of using the elastic portion 304 in which the restoring force F_{K} has the linear characteristic. When a coil spring having a non-linear characteristic or the like is used as the elastic portion 304, it is possible to fit a curve with a displacement curve of the attraction force F_{M} by the magnetic force of the magnetic body portion 306. Consequently, it is possible to further reduce the force necessary for the DEA used for the driving unit 305, and as a result, this can contribute to downsizing the dimensions of the actuator unit 102 (particularly, in the direction orthogonal to the longitudinal direction).

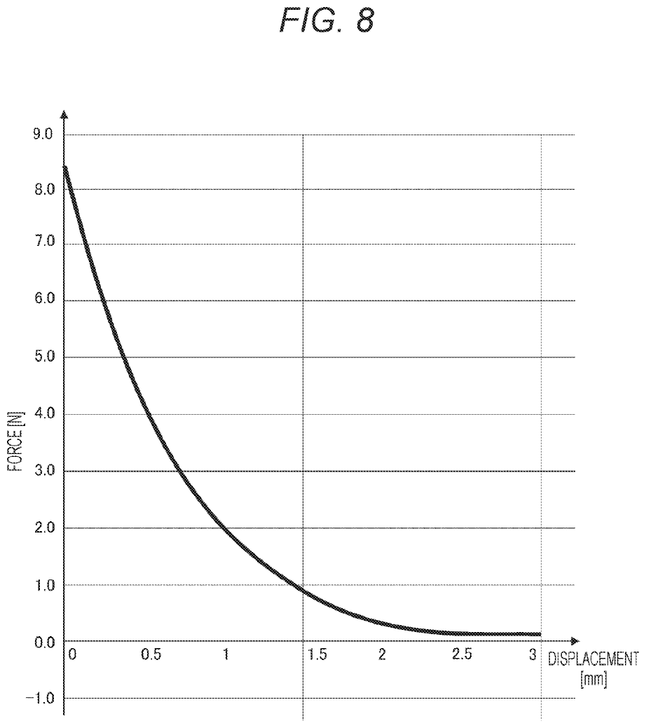

When the driving unit 305 is contracted in the linear movement direction indicated by the arrow A, the sum of the attraction force F_{M} by the magnetic force of the magnetic body portion 306 and the generative force F_{DEA} of the driving unit 305 becomes the gripping force. FIG. 8 illustrates exemplary calculation value of the gripping force of the gripping portion 101 when the actuator unit 102 displaces the acting portion 301 in the linear movement direction indicated by the arrow A. Note that a horizontal axis represents the displacement amount of the acting portion 301, a maximum displacement is set to 3 mm, and a vertical axis represents the force [N]. Furthermore, the maximum displacement amount of the actuator unit 102 is set to 3 mm, the position where the acting portion 301 is displaced maximally in the linear movement direction indicated by the arrow A (see FIG. 4) is set to 0 on the horizontal axis, and the opposite direction of the arrow A is defined as the positive direction of the horizontal axis. Additionally, calculation is made on the basis of the calculation results illustrated in FIG. 7 while setting the generative force F_{DEA} of the driving unit 305 to less than 1 N (that is, F_{DEA}<1 [N]). As illustrated, the gripping force is transitional together with the displacement amount of the actuator unit 102.

The sum of the attraction force F_{M} by the magnetic force of the magnetic body portion 306 and the generative force F_{DEA} of the driving unit 305 becomes the traction force by the actuator unit 102, and it is found from FIG. 8 that force of 7N or more can be obtained. It should be fully understood that force minimally required for the driving unit 305 including the DEA can be reduced to 1 N or less by compensation with the restoring force Fₖ of the elastic portion 304 including the coil spring or the like. Therefore, the output of the DEA can be suppressed small and it is possible to downsize the dimensions of the actuator unit 102 (particularly, in the direction orthogonal to the longitudinal direction).

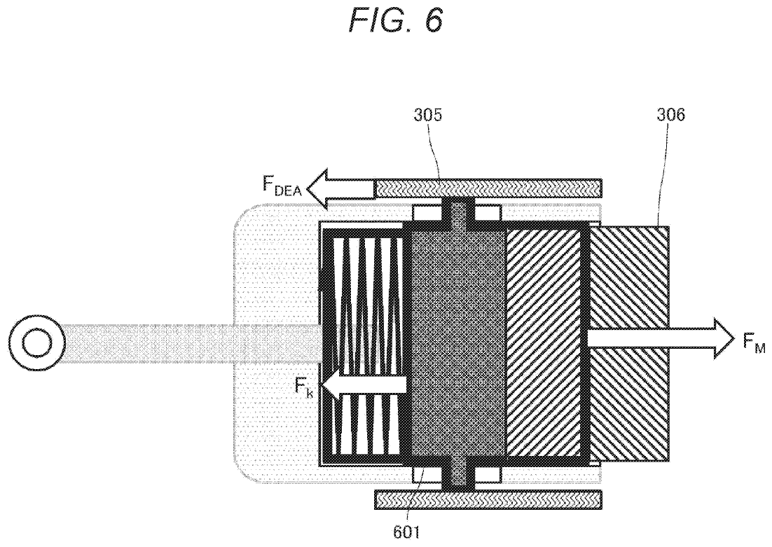

Furthermore, FIG. 6 illustrates the force acting on the second system when the gripping portion 101 is opened to release the gripped object. In the drawing, the components constituting the second system are surrounded by a thick line 601 (the second system includes the sliding portion 303, the second magnetic body 307, and the elastic portion 304 as described above). When the resultant force F2 of the force acting on the second system acts in the opposing direction of the arrow A, the resultant force becomes the force that pulls away, from the magnetic body portion 306, the second magnetic body portion 307 integrated with the sliding portion 303, and the gripping portion 101 can be opened by making the second system slide.

The sliding portion 303 is applied with: the restoring force Fₖ from the elastic portion 304; the driving force F_{DEA} by the driving unit 305 (note, however, when the DEA is extended); and the attraction force F_{M} by which the second magnetic body portion 307 attached to the other end surface of the sliding portion 303 is sucked by the magnetic force of the magnetic body portion 307. Among these kinds of force, the restoring force Fₖ and the driving force F_{DEA} acts in the direction opposite to the traction force indicated by the arrow A (note, however, when DEA is extended), and the attraction force F_{M} by the magnetic force of the magnetic body portion 306 acts in the direction same as the traction force indicated by the arrow A. Therefore, the resultant force F₂ acting on the second system is as represented by Expression (2) below.

[Math. 2]

F₂=F_{DEA}+Fₖ−F_{M} (2)

When F₂>0, that is, when the sum of the restoring force Fₖ and the driving force F_{DEA} is larger than the magnetic force F_{M}, in other words, when the driving force F_{DEA} is larger than a difference between the magnetic force F_{M} and the restoring force Fₖ, the second magnetic body portion 307 integrated with the sliding portion 303 is pulled away from the magnetic body portion 306, and the gripping portion 101 can be opened by making the second system slide. A conditional expression of pulling away the second magnetic body portion 307 from the magnetic body portion 306 is as represented by Expression (3) below.

[Math. 3]

F_{DEA}>F_{M}−Fₖ (3)

Therefore, when the elastic portion (coil spring) 304 is selected so as to obtain appropriate restoring force Fₖ, the second magnetic body portion 307 can be pulled away from the magnetic body portion 306 with the small driving force F_{DEA} of the driving unit 305 including the DEA, and the grip lock can be released.

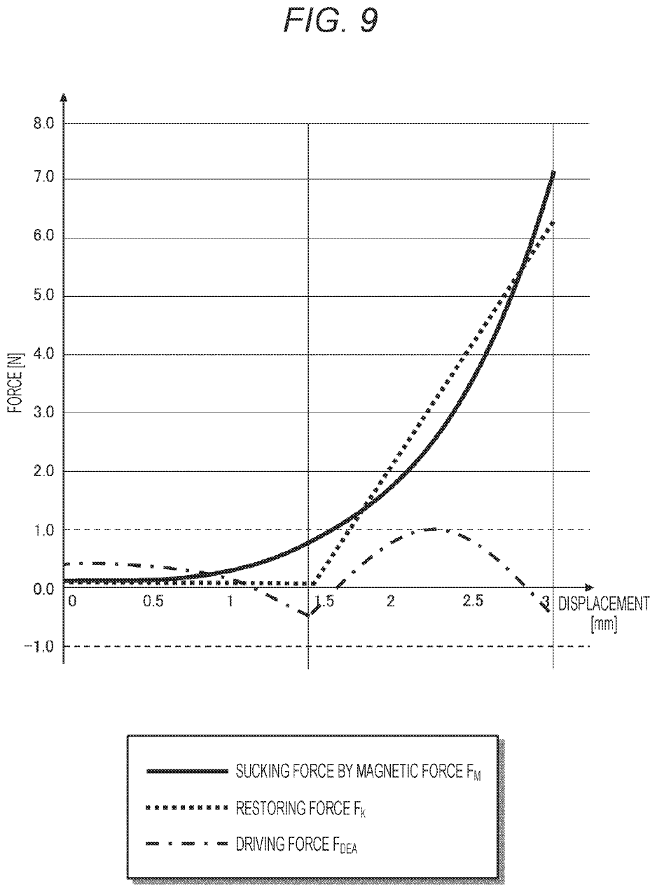

FIG. 9 illustrates exemplary calculation values of the attraction force F_{M} by the magnetic force of the magnetic body portion 306, the restoring force F_{K} of the elastic portion 304, and the generative force F_{DEA} of the driving unit (DEA) 305 when the actuator unit 102 displaces the acting portion 301 in the opposite direction of the arrow A (that is, when the gripping portion 101 is opened). Note that a horizontal axis represents the displacement amount of the acting portion 301, a maximum displacement is set to 3 mm, and a vertical axis represents the force [N]. Furthermore, the maximum displacement amount of the actuator unit 102 is set to 3 mm, the position where the acting portion 301 is displaced maximally in the linear movement direction indicated by the arrow A (see FIG. 4) is set to 0 on the horizontal axis, and the opposite direction of the arrow A is defined as the positive direction of the horizontal axis. Additionally, the calculation is made while setting an elastic coefficient of the elastic portion 304 as k=4.5 N/mm.

The attraction force F_{M} by the magnetic force of the magnetic body portion 306 attenuates in inverse proportion to the distance from the second magnetic body portion 307. Furthermore, the elastic portion 304 includes the coil spring having, for example, the linear characteristic, and the restoring force F_{K} thereof is decreased in proportion to the distance from where the displacement amount is close to 1.5 mm. Therefore, the more the displacement amount is increased and the larger the open/close angle of the gripping portion 101 is, the more the gripping force is nonlinearly reduced. Furthermore, the restoring force F_{K} of the elastic portion 304 has the linear characteristic, and a magnitude relation with the attraction force F_{M} by the magnetic force of the magnetic body portion 306 is reversed in the process in which the acting portion 301 is displaced, but an insufficient force is compensated by the generative force F_{DEA} of the driving unit 305. It is found that when the generative force F_{DEA} of the driving unit 305 is in a range of −1 to +1 [N], the actuator unit 102 is operable.

A leftmost end of the horizontal axis of the graph illustrated in FIG. 9 is the maximum displacement position of the actuator unit 102 where the magnetic body portion 306 closely contacts (or is positioned closest to) the second magnetic body portion 307. As already described with reference to FIG. 7, since the attraction force F_{M} by the magnetic force of the magnetic body portion 306 becomes larger than the restoring force F_{K} of the elastic portion 304 at the maximum displacement position of the actuator unit 102, the gripping portion 101 is brought into the grip lock state by stopping the driving force F_{DEA} of the driving unit 305. Therefore, when the driving unit 305 supplies the driving force F_{DEA} larger than the difference between the magnetic force F_{M} and the restoring force Fₖ, the grip lock of the gripping portion 101 can be released.

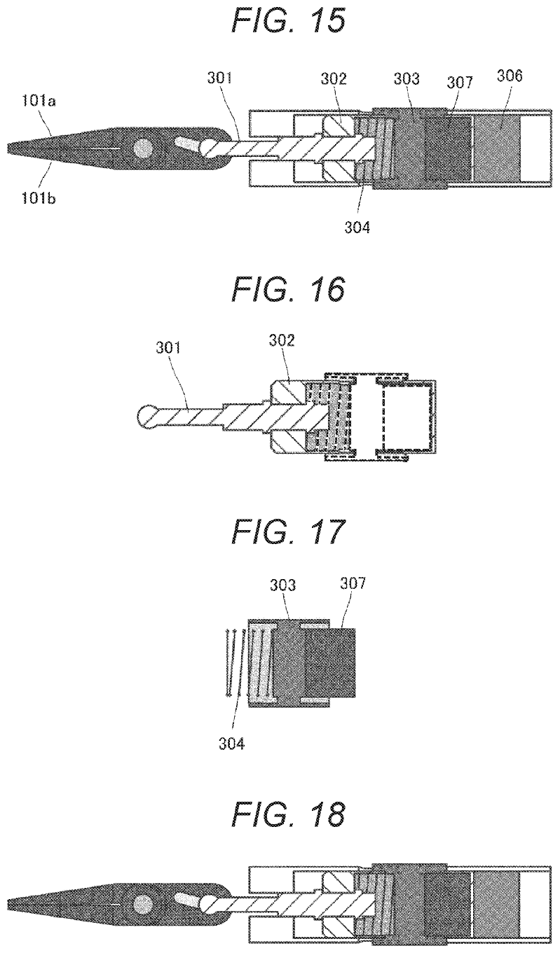

FIG. 15 illustrates exemplary implementation of the actuator unit 102. Furthermore, FIG. 16 illustrates a portion of the first system of the actuator unit 102 in an extracted manner, and FIG. 17 illustrates a portion of the second system thereof in an extracted manner. The first system illustrated in FIG. 16 includes the supporting portion 302 that supports the acting portion 301. The supporting portion 302 is movable in the linear movement direction (left direction in the drawing paper of FIG. 16) of the actuator unit 102 indicated by the arrow A in FIG. 1 and in the opposite direction thereof. Furthermore, the second system illustrated in FIG. 17 includes the sliding portion 303, the elastic portion 304, and the second magnetic body portion 307. The second magnetic body portion 307 moves the first system illustrated in FIG. 16 in the linear movement direction by the magnetic force generated between the second magnetic body portion 307 and the magnetic body portion 306. Furthermore, the elastic portion 304 can apply the force to the first system in the opposite direction of the linear movement direction. The sliding portion 303 has one surface (end surface on the distal end side) that is oriented in a direction parallel to the linear movement direction and connected to the elastic portion 304, and has the other surface (end surface on the proximal end side) connected to the second magnetic body portion 307. The sliding portion 303 can be relatively moved in the direction parallel to the linear movement direction by the driving of the driving unit 305 (not illustrated in FIGS. 15 to 17).

Furthermore, FIGS. 18 to 25 illustrate how the gripping portion 101 is changed from the closed state to the opened state and again changed to the closed state by the operation of the actuator unit 102.

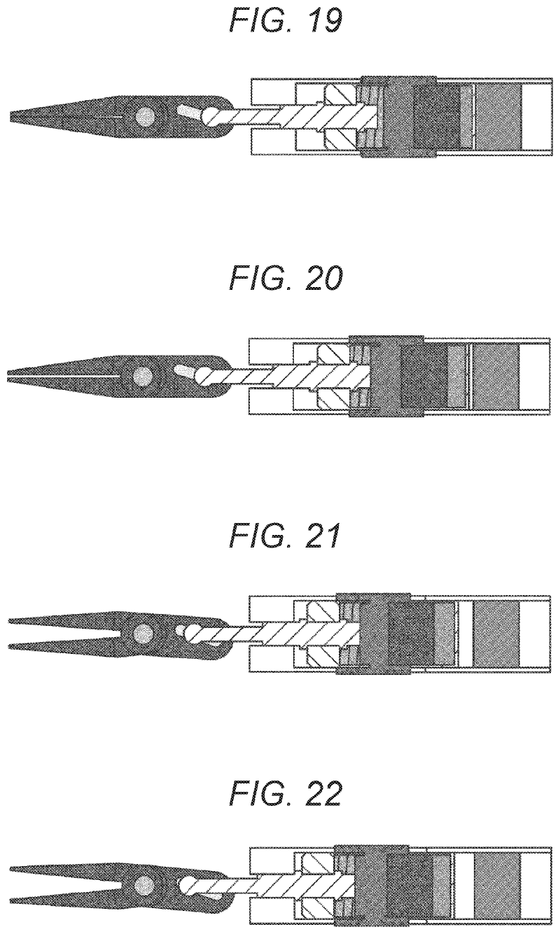

FIGS. 18 to 22 each illustrate how the gripping portion 101 is opened by linear movement operation of the actuator unit 102 toward the left side in the drawing paper. During steps between FIGS. 18 and 19, the driving unit 305 is extended, the second magnet portion 307 is separated from the magnet portion 306 by the resultant force of tensile force Fₖ of the elastic portion 304 and the driving force F_{DEA} of the driving unit 305, and the second system starts linear movement toward the left side in the drawing paper.

Then, when the end surface of the sliding portion 303 abuts on a rear end portion of the acting portion 301 at a time point illustrated in FIG. 20, the first system and the second system are integrally and linearly moved toward the left side in the drawing paper during the steps between FIGS. 20 to 22, and as a result, the gripping portion 101 can be opened as illustrated in FIG. 22.

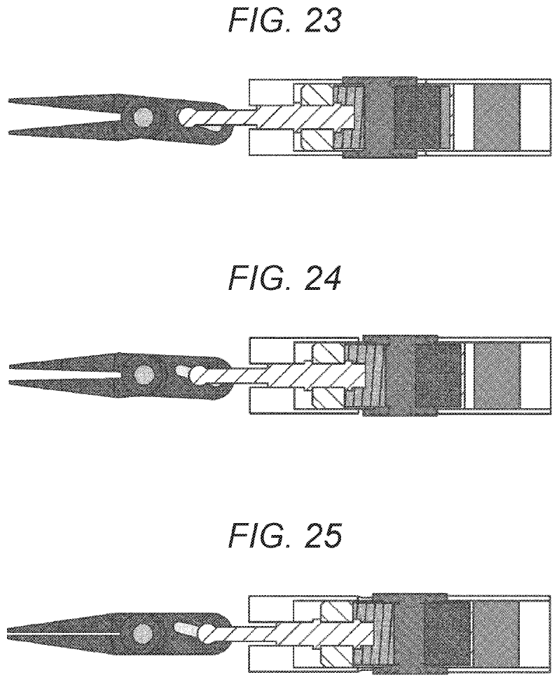

FIGS. 22 to 25 illustrate how the gripping portion 101 is by linearly moving the actuator unit 102 to the right side in the drawing paper and generating the traction force. In the state illustrated in FIG. 22, when the driving unit 305 stops the driving force F_{DEA} or switches to the driving force F_{DEA} directed to the right side in the drawing paper (namely, the magnet portion 306), influence of the sucking force F_{M} by which the magnet portion 306 sucks the second magnet portion 307 with the magnetic force is increased, and the second system starts the linear movement toward the right side in the drawing paper as illustrated in FIG. 23.

In steps during FIGS. 24 and 25, the end surface of the sliding portion 303 is separated from the rear end portion of the acting portion 301, and only the second system is moved toward the right side in the drawing paper. Furthermore, when the coil spring as the elastic portion 304 exceeds the natural length, the elastic force Fₖ is applied to the second system toward the left side in the drawing paper, but the attraction force F_{M} by the magnetic force of the magnet portion 306 is stronger, and therefore, the second system keeps movement toward the right side in the drawing paper.

Then, as illustrated in FIG. 25, the gripping portion 101 is completely closed at the maximum displacement position where the second magnet portion 307 is adsorbed to the magnet portion 306. At this maximum displacement position, the gripping portion 101 can be made into the grip lock state by selecting the coil spring used for the elastic portion 304 such that the attraction force F_{M} by the magnetic force of the magnetic body portion 306 becomes larger than the restoring force F_{K} of the elastic portion 304.

As described above, according to the actuator unit 102 according to the present embodiment, the large traction force can be generated when the open/close angle of the gripping portion 101 is close to zero degrees. Therefore, the gripping portion 101 can grasp a needle and living tissue with strong force during surgery. In contrast, when the open/close angle of the gripping portion 101 is fixed around zero degrees due to a structural failure or the like, the body tissue is kept gripped, which is dangerous. Accordingly, it is preferable that the actuator unit 102 be equipped with a structure for security assurance.

As an example, the magnetic body portion 306 on the proximal end side may have a detachable structure. Specifically, as indicated by reference sign 311 in FIG. 4, a wire is attached to the end surface on the proximal end side of the magnetic body portion 306 such that the magnetic body portion 306 can be dropped (or can be pulled away manually from the second magnetic body portion 307) by pulling this emergency wire 311. Consequently, the traction force of the actuator unit 102 is lost, and the gripping portion 101 is opened and the gripped object can be released.

Furthermore, in a case of using an electromagnet including a coil as the magnetic body portion 306 instead of a permanent magnet (as described above), the direction of the magnetic force can be changed to the opposing direction by changing a direction of coil current, and the grip lock can be easily released. Furthermore, in the event of structural failure or emergency also, a polarity of the electromagnet is switched to release the grip lock, and the gripped object can be released. In the event of electrical failure, the magnetic force is lost by stopping the current to the coil, and therefore, the grip lock is automatically released.

Subsequently, the force sensor 103 applied to the surgical robot 100 illustrated in FIG. 1 will be described in detail. In the present embodiment, the force sensor 103 is arranged in the region located between the actuator unit 102 and the proximal end and free from acting of the traction force to generate the gripping force of the gripping portion 101 (see FIG. 1). Therefore, since the traction force of the actuator unit 102 does not interfere with the external force applied in the long axis direction of the end effector, the sensitivity of the force sensor 103 is not degraded, and a detection signal from the force sensor 103 can be easily calibrated.

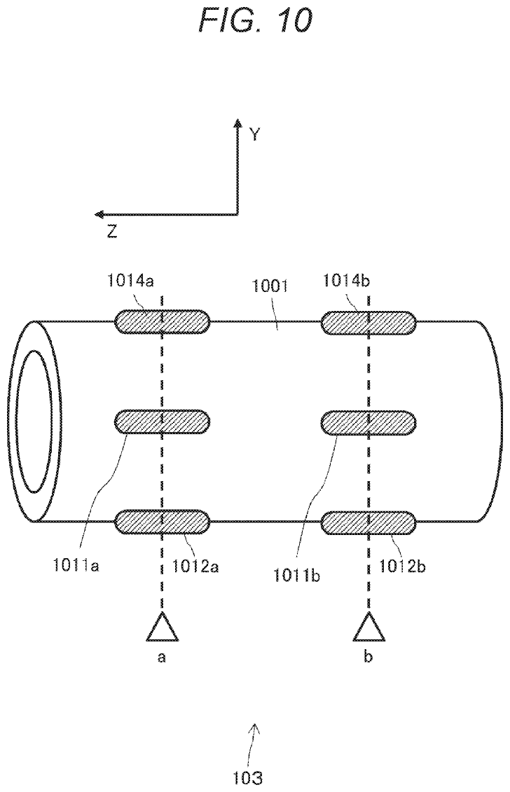

FIG. 10 illustrates an exemplary configuration of the force sensor 103. The illustrated force sensor 103 includes: a strain element 1001 having a hollow cylindrical shape; and strain detection element(s) disposed at one or more places on an outer periphery of the strain element 1001. Note, however, that a part of a link structure included in the surgical robot 100 can also be used as the strain element 1001.

In the example illustrated in FIG. 10, a plurality of strain detection elements for detecting strain in XY directions at the respective different two positions a and b in the long axis direction is attached to the outer periphery of the strain element 1001. Specifically, at the position a, a pair of strain detection elements 1011a and 1013a (not illustrated in FIG. 10) to detect a strain amount in the X direction of the strain element 1001 are attached to facing sides of the outer periphery of the strain element 1001. Furthermore, a pair of strain detection elements 1012a and 1014a to detect a strain amount in the Y direction of the strain element 1001 are attached to facing sides of the outer periphery of the strain element 1001. Similarly, at the position b, a pair of strain detection elements 1011b and 1013b (not illustrated in FIG. 10) to detect the strain amount in the X direction of the strain element 1001 are attached, and also a pair of strain detection elements 1012b and 1014b to detect a strain amount in the Y direction are attached.

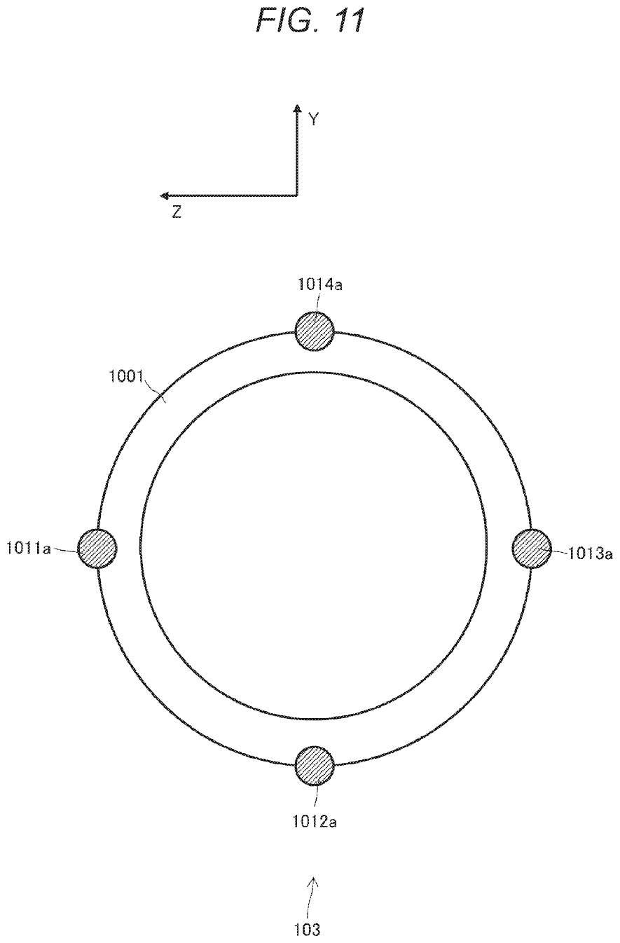

FIG. 11 is a view illustrating an XY cross section at the position a of the strain element 1001. As is clear from the drawing, the pair of strain detection elements 1011a and 1013a that detect the strain amount in the X direction are attached to the facing sides in the X direction of the outer periphery of the strain element 1001, and the pair of strain detection elements 1012a and 1014a that detect the strain amount in the Y direction are attached to the facing sides in the Y direction of the outer periphery of the strain element 1001. Note that, as for the XY cross section at the position b of the strain element 1001 also, the pair of strain detection elements 1011b and 1013b that detect the strain amount in the X direction are attached to the facing sides in the X direction of the outer periphery of the strain element 1001, and the pair of strain detection elements 1012b and 1014b that detect the strain amount in the Y direction are attached to the facing sides in the Y direction of the outer periphery of the strain element 1001 in a manner similar to FIG. 11, although not illustrated.

First, a description will be provided referring to FIG. 12 for a reason why the pair of strain detection elements 1011a and 1013a (or 1011b and 1013b) are disposed on the facing sides in the X direction and the pair of strain detection elements 1012a and 1014a (or 1012b and 1014b) are disposed on the facing sides in the Y direction at the one detecting position.

As illustrated in FIG. 12(A), in a case where only one strain detection element 1211 is attached to a cantilever beam 1201, the strain detection element 1211 is compressed when Z-direction external force F_{z} is applied to the cantilever beam 1201, and therefore, the external force F_{z} can be measured. However, since the strain detection element 1211 is stretched even if the cantilever beam 1201 is bent in either an upper direction or a lower direction in the drawing paper, it is not possible to identify which one of directions, a positive direction or a negative direction (upper or lower direction in the drawing paper) an acting direction of the external force F_{y} applied in the Y direction is.

In contrast, as illustrated in FIG. 12(B), in a case of attaching a pair of detection elements 1221 and 1222 on facing sides in the Y direction of the cantilever beam 1201, when the cantilever beam 1201 is bent upward in the drawing paper, the strain detection element 1221 on one side is compressed and the strain detection element 1222 on the other side is stretched, whereas when the cantilever beam 1201 is bent downward in the drawing paper, the strain detection element 1221 on the one side is stretched and the strain detection element 1222 on the other side is compressed. Therefore, it is possible to identify the acting direction of the external force F_{y} applied in the Y direction on the basis of the relation between the positive and negative signs of strain amounts detected by the pair of detection elements 1221 and 1222 attached to the facing sides in the Y direction.

Accordingly, it is possible to detect the Z-direction external force acting on the strain element 1001 by acquiring the sum of the respective strain amounts detected by the pair of strain detection elements 1011a and 1013a (or 1011b and 1013b) attached to the facing sides in the X direction at an arbitrary position in the long axis direction of the strain element 1001, and also it is possible to calculate X-direction external force acting on the strain element 1001 by acquiring a difference between the respective strain amounts.

Furthermore, the strain amount detected by each of the strain detection elements 1011a and 1013a (or 1011b and 1013b) includes not only a component caused by acting force but also a component caused by a temperature change, but there are advantages that the component caused by the temperature change is setoff at the time of calculating the X-direction external force by acquiring the difference between the respective strain amounts, and it is not necessary to perform temperature compensation processing. Note that a method of performing the temperature compensation by acquiring a detection value difference between sensors installed on facing sides, for example, a 4-gauge method using four strain gauges is known in the industry.

Similarly, it is possible to detect Z-direction external force acting on the strain element 1001 by acquiring the sum of the respective strain amounts detected by the pair of strain detection elements 1012a and 1014a (or 1012b and 1014b) attached to the facing sides in the Y direction at an arbitrary position in the long axis direction of the strain element 1001, and also it is possible to calculate the Y-direction external force acting on the strain element 1001 by acquiring a difference between the respective strain amounts. Furthermore, the strain amount detected by each of the strain detection elements 1012a and 1014a (or 1012b and 1014b) includes not only a component caused by acting force but also a component caused by a temperature change, but there are advantages that the component caused by the temperature change is setoff at the time of calculating the Y-direction external force by acquiring the difference between the respective strain amounts, and it is not necessary to perform the temperature compensation processing (same as described above).

Next, a description will be provided for a reason for adopting the configuration in which the strain amounts in the XY directions are detected at the different two positions a and b in the long axis direction of the strain element 100.

It is possible to calculate translational force from a strain amount at one place of the cantilever beam, but it is not possible to calculate a moment. In contrast, it is possible to calculate both a moment and the translational force from strain amounts at two or more places. Therefore, according to the configuration illustrated in FIG. 10, X-direction translational force Fₓ acting on the strain element 1001 and a moment Mₓ around the X axis can be calculated on the basis of the X-direction strain amount detected at the two positions a and b, and similarly, Y-direction translational force F_{y} acting on the strain element 1001 and a moment M_{y} around the Y axis can be calculated on the basis of the Y-direction strain amounts detected at the two positions a and b. Therefore, it can be said that the force sensor 103 is equipped with a sensor having 4 degrees of freedom (DOF) of the moments Mₓ and M_{y} around the two axes in addition to the two-direction translational force Fₓ and F_{y}.

In FIG. 10 and FIG. 11, the strain element 1001 is illustrated to have a simple cylindrical shape to simplify the drawings. When the strain element 1001 has a structure suitable as a strain element, the detection performance as the 4DOF sensor is improved. That is, in a case where the strain element 1001 is formed in a shape in which stress is concentrated at each of the two measurement positions a and b in the long axis direction and the strain element 1001 is easily deformed, the strain amounts can be easily measured by the strain detection elements 1011a to 1014a and 1011b to 1014b, and improvement in the detection performance as the 4DOF sensor is expected.

Furthermore, as the strain detection element, a capacitive sensor, a semiconductor strain gauge, a foil strain gauge, and the like are also widely known in this industry, and any of these can be used as the strain detection elements 1011a to 1014a and 1011b to 1014b. Note, however, that in present embodiment, a fiber Bragg grating (FBG) sensor manufactured by utilizing optical fibers are used as the strain detection elements 1011a to 1014a and 1011b to 1014b.

Here, the FBG sensor is a sensor formed by engraving diffraction gratings (gratings) along a long axis of each optical fiber, and it is possible to detect, as a wavelength change in reflection light relative to incident light in a predetermined wavelength band (Bragg wavelength), a change in an interval between the diffraction gratings caused by expansion or contraction along with a change in a strain or a temperature caused by the acting force. Then, the wavelength change detected from the FBG sensor can be converted into a strain, stress, and a temperature change which are to be causes. Since the FBG sensor utilizing the optical fibers has a small transmission loss (hardly carries noise from the outside), detection accuracy can be kept high even under an assumed usage environment. Furthermore, the FBG sensor has advantages of easily coping with sterilization necessary for medical care and coping with a strong magnetic field environment.

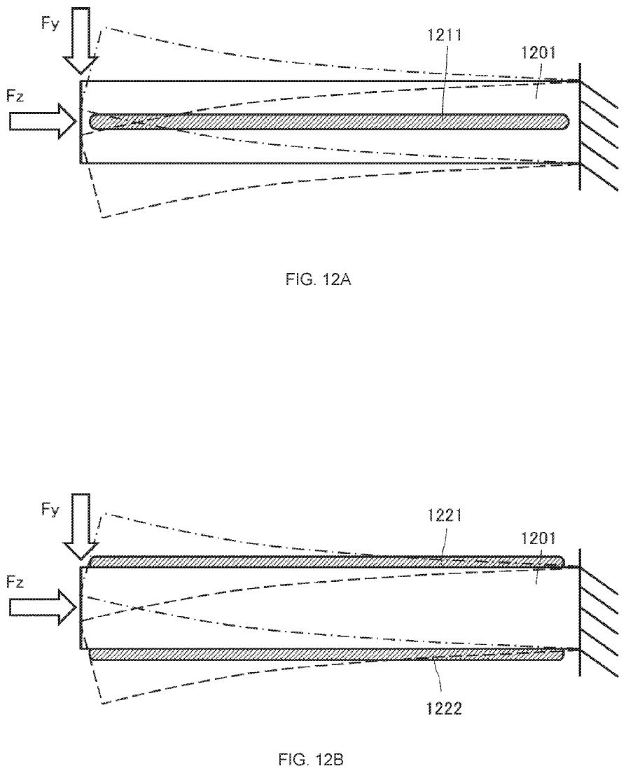

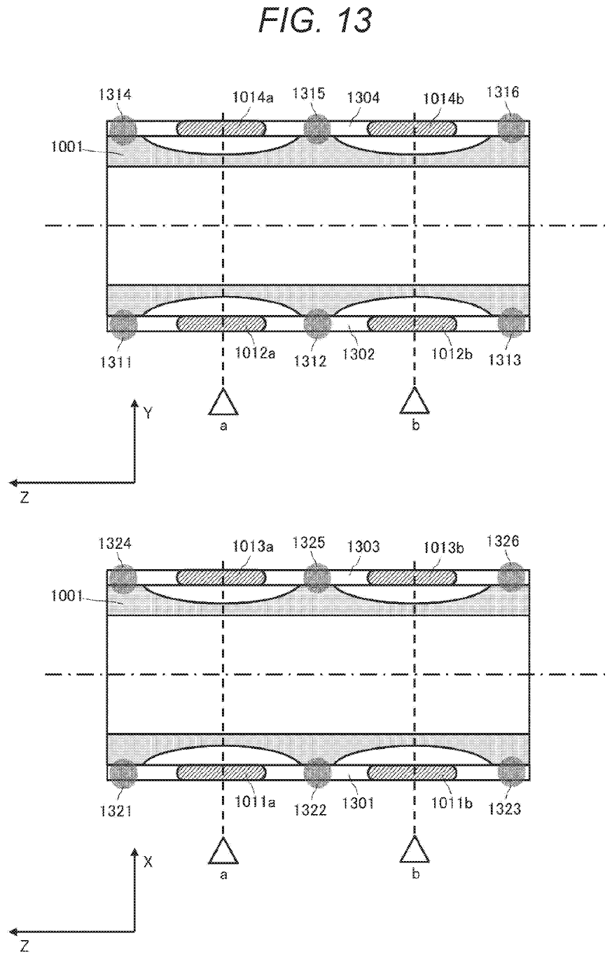

A description will be provided with reference to FIG. 13 for a structure of the strain element 1001 that can be easily deformed at the two measurement positions a and b, and a method of installing, on the outer periphery of the strain element 1001, the strain detection elements 1011a to 1014a and 1011b to 1014b utilizing the FBG sensors.

FIG. 13 illustrates a YZ cross section and a ZX cross section of the strain element 1001 respectively. In the drawing, the YZ cross section and the ZX cross section of the strain element 1001 are colored in gray. The strain element 1001 is, for example, hollow and has a rotationally symmetric shape around the long axis. The strain element 1001 has a structure in which a recess having a radius gradually reduced is provided in each of the different two measurement positions a and b in the long axis direction. Therefore, when force acts in at least one of directions X or Y, stress is concentrated at each of the two measurement positions a and b, and the strain element 1001 is easily deformed and can be used as a strain element.

The strain element 1001 is manufactured by using, for example, stainless steel (steel use stainless: SUS), a Co—Cr alloy, or a titanium material which are known as metal materials excellent in biocompatibility. For example, from a viewpoint of forming a strain element in a partial structure of the surgical robot 100, it is preferable to manufacture the strain element 1001 using a material having mechanical characteristics, for example, a titanium alloy. The acting force to the end effector such as the gripping portion 101 can be measured with high sensitivity by using the low-rigidity material for the strain element 1001. Furthermore, the titanium alloy is biocompatible and is also a preferable material in a case of use in medical practice such as a surgical operation.

A pair of optical fibers 1302 and 1304 are laid in the long axis direction on the facing sides in the Y direction of the outer periphery of the strain element 1001. Similarly, a pair of optical fibers 1301 and 1303 are laid in the long axis direction on the facing sides in the X direction of the outer periphery of the strain element 1001. In short, the four optical fibers 1301 to 1304 are laid in the entire strain element 1001.

The optical fibers 1302 and 1304 laid on the facing sides in the Y direction have ranges that overlap with the two recesses of the strain element 1001 (or in the vicinity of the measurement positions a and b) and have the FBG sensors formed by engraving the diffraction gratings, and the FBG sensors are utilized as the strain detection elements 1012a, 1012b, 1014a, and 1014b, respectively. The portions including the FBG sensors in the optical fibers 1302 and 1304 are indicated by hatching in the drawing.

Furthermore, the respective optical fibers 1302 and 1304 are fixed to the outer periphery of the strain element 1001 with an adhesive or the like at both ends 1311 to 1313 and 1314 to 1316 of the portions including the FBG sensors 1012a, 1012b, 1014a, and 1014b on the surface of the strain element 1001. Therefore, when the external force acts and bends the strain element 1001 in the Y direction, the respective optical fibers 1302 and 1304 are also integrally deformed, and the portions of the FBG sensors, namely, the strain detection elements 1012a, 1012b, 1014a, and 1014b are strained.

Similarly, the optical fibers 1301 and 1303 laid on the facing sides in the X direction have ranges that overlap with the two recesses of the strain element 1001 (or in the vicinity of the measurement positions a and b) and have the FBG sensors formed by engraving the diffraction gratings, and the FBG sensors are utilized as the strain detection elements 1011a, 1011b, 1013a, and 1013b, respectively. The portions including the FBG sensors in the optical fibers 1301 and 1303 are indicated by hatching in the drawing.

Furthermore, the respective optical fibers 1301 and 1301 are fixed to the outer periphery of the strain element 1001 with an adhesive or the like at both ends 1321 to 1323 and 1324 to 1326 of the portions including the FBG sensors 1011a, 1011b, 1013a, and 1013b on the surface of the strain element 1001. Therefore, when the external force acts and bends the strain element 1001 in the Y direction, the respective optical fibers 1301 and 1303 are also integrally deformed, and the portions of the FBG sensors, namely, the strain detection elements 1011a, 1011b, 1013a, and 1013b are strained.

In FIG. 13, only the portions attached to the outer periphery of the strain element 1001 are illustrated out of the optical fibers 1301 to 1304 used as the strain detection elements 1011a to 1014a and 1011b to 1014b, and other portions are not illustrated. Actually, it should be understood that each of these optical fibers 1301 to 1304 have a total length of, for example, about 400 millimeters and extend to a detection unit and a signal processing unit (both not illustrated).

The detection unit and the signal processing unit are disposed apart from the end effector, for example, in the vicinity of a base of the surgical robot 100. The detection unit makes light of a predetermined wavelength (Bragg wavelength) enter each of the optical fibers 1301 to 1304, receives reflection light thereof and detects a wavelength change AA. Then, the signal processing unit calculates two-direction translational force Fₓ and F_{y} and two-direction moments Mₓ and M_{y} acting on the gripping portion 101 on the basis of wavelength changes detected from the respective FBG sensors provided as the strain detection elements 1011a to 1014a and 1011b to 1014b respectively attached to the facing sides in each of the XY directions of the strain element 1001.

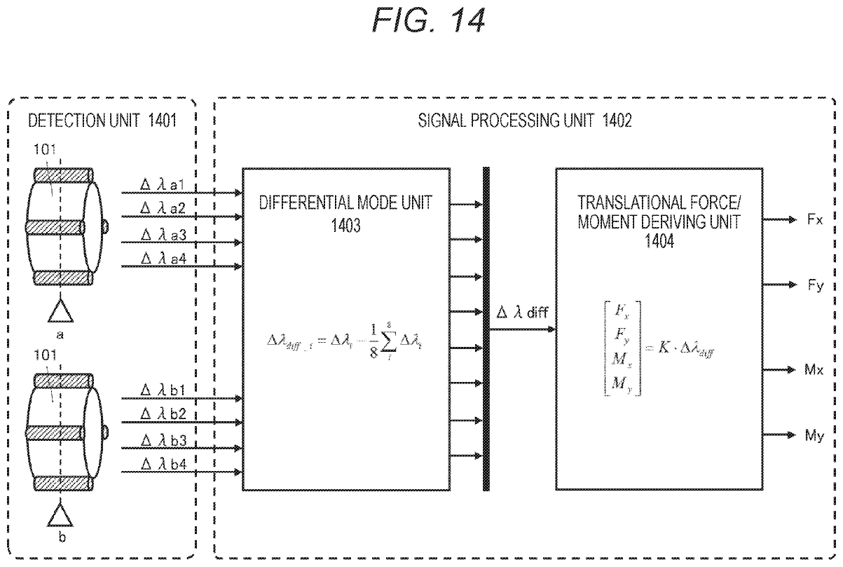

FIG. 14 schematically illustrates a processing algorithm for the 4DOF sensor to calculate, in a detection unit 1401 and a signal processing unit 1402, the two-direction translational force Fₓ and F_{y} and the two-direction moments Mₓ and M_{y} acting on the gripping portion 101 provided as the end effector, on the basis of detection results obtained from the FBG sensors respectively formed on the optical fibers 1301 to 1304 laid in the strain element 1001.

The detection unit 1401 detects, on the basis of the reflection light of the incident light in the predetermined wavelength band to each of the optical fibers 1301 to 1304 attached to the respective facing sides in the respective XY directions of the strain element 1001, respective wavelength changes AAa1 to AAa4 in the respective FBG sensors as the strain detection elements 1011a to 1014a disposed at the position a of the strain element 1001 in a case where the translational force Fₓ and F_{y} and moments Mₓ and M_{y} act. Note, however, that each of the detected wavelength changes AAa1 to AAa4 also include a wavelength change component caused by a temperature change.

Furthermore, the detection unit 1401 detects, on the basis of the reflection light of the incident light in the predetermined wave length to each of the optical fibers 1301 to 1304 attached to the respective facing sides in the respective XY directions of the strain element 1001, respective wavelength changes AAb1 to AAb4 in the respective FBG sensors as the strain detection elements 1011b to 1014b disposed at the position b of the strain element 1001 in a case where the translational force Fₓ and F_{y} and moments Mₓ and M_{y} act. Note, however, that each of the detected wavelength changes AAb1 to AAb4 also includes a wavelength change component caused by a temperature change.

Here, the wavelength changes AAa1 to AAa4 detected by the detection unit 1401 from the respective optical fibers 1301 to 1304 at the position a are respectively equivalent to strain amounts Δεa1 to Δεa4 generated at the position a of the strain element 1001 when the translational force Fₓ and F_{y} and moments Mₓ and M_{y} act. Furthermore, the wavelength changes Δλb1 to Δλb4 detected by the detection unit 1401 from the optical fibers 1301 to 1304 at the position b are respectively equivalent to strain amounts Δεb1 to Δεb4 generated at the position b of the strain element 1001 when the translational force Fₓ and F_{y} and moments Mₓ and M_{y} act (note, however, that this is a case where a wavelength change component caused by a temperature change is ignored).

A differential mode unit 1403 subtracts an average value of these eight inputs from each of the above-described eight inputs Δλa1 to Δλa4 and Δλb1 to Δλb4 received from the detection unit in accordance with Expression (4) below, and outputs obtained values to a latter translational force/moment deriving unit 1404. Each of the wavelength changes detected at the respective positions a and b includes a wavelength change component Δλₜₑₘₚ caused by a temperature change together with a wavelength change component caused by strain due to action of translational force Fₓ and F_{y} and the moments Mₓ and M_{y}. The differential mode unit 1403 can cancel the wavelength change component Δλₜₑₘₚ caused by the temperature change.

$$\begin{matrix}[ Math . 4 ] & \\ Δ {λ}_{diff\_i} = Δ {λ}_{i} - \frac{1}{8} {{\sum }_{i}}^{8} Δ {λ}_{i} & ( 4 )\end{matrix}$$

Then, the translational force/moment deriving unit 1404 multiplies Δλ_{diff} received from the differential mode unit 1403 by a calibration matrix K as represented by Expression (5) below and calculates the translational force Fₓ and F_{y} and the moments Mₓ and M_{y}.

$$\begin{matrix}[ Math . 5 ] & \\ [ \begin{matrix}{F}_{x} \\ {F}_{y} \\ {M}_{x} \\ {M}_{y}\end{matrix} ] = K \cdot Δ {λ}_{diff} & ( 5 )\end{matrix}$$

Note that the signal processing unit 1402 illustrated in FIG. 14 and the calibration matrix K used in the calculation in Expression (5) can be derived from, for example, a calibration experiment. In the present embodiment, the force sensor 103 is arranged in the region located between the actuator unit 102 and the proximal end and free from acting of the traction force to generate the gripping force of the gripping portion 101 (see FIG. 1). Therefore, since the traction force of the actuator unit 102 does not interfere with the external force applied in the long axis direction of the end effector, the calibration matrix can be easily calculated.

For example, in a case where the surgical robot 100 operates as a slave device in the master-slave robot system, a detection result from the force sensor 103 of the 4DOF described above is transmitted to a master device as feedback information in response to remote control. The feedback information can be utilized on the master device side for various purposes. For example, the master device can perform force sense presentation for an operator on the basis of the feedback information from the slave device. This presentation can contribute to achievement of minimal invasive endoscopic treatment.

### INDUSTRIAL APPLICABILITY

As described above, the technology disclosed in the present specification has been described in detail with reference to the specific embodiment. However, it is obvious that those skilled in the art can make modifications and substitutions of the embodiment without departing from the scope of the technology disclosed in the present specification.

The application range of the actuator device and the end effector proposed in the present specification is not limited to the gripping purpose. For example, the large gripping force can be generated with small traction force by applying the actuator device and the end effector proposed in the present specification to various situations where large gripping force is desired to be obtained when an open/close angle is small, such as a stationery (scissors or clips) and a work tool (piers or nippers).

Furthermore, the embodiment related to the end effector to which the pair of surgical forceps including the pair of blades coupled in the openable/closable manner is applied has been mainly described in the present specification, but the application range of the technology disclosed in the present specification is not limited thereto. As the end effector, not only the forceps but also a medical instrument such as a pair of tweezers or a cutting instrument that contacts a patient during a surgical operation, or an imaging device such as an endoscope or a microscope may be attached. Furthermore, the pressurizing portion is not limited to the elastic member as far as force in the opposite direction of the predetermined direction can be applied. For example, a magnet that generates attraction force in the opposite direction may be used.

In short, the technology disclosed in the present specification has been described with the embodiment as exemplified, but the content of the present specification should not be interpreted in a limited manner. The scope of the technology disclosed in the present specification should be determined in consideration of the claims.

Note that the technology disclosed in the present specification can also have the following configurations.

(1) An actuator device including:

a first magnetic body portion;

a first system movable in a predetermined direction or an opposite direction of the predetermined direction;

a second system including a second magnetic body portion that moves the first system in the predetermined direction by magnetic force generated between the second magnetic body portion and the first magnetic body portion, and a pressurizing portion capable of applying, to the first system, force in the opposite direction of the predetermined direction; and

a driving unit capable of applying, to the second system, force in the predetermined direction or the opposite direction by driving.

(2) The actuator device recited in (1) above, in which

the pressurizing portion includes an elastic portion.

(3) The actuator device recited in (2) above, in which

the more the first system is drawn in the predetermined direction, the more the force in the opposite direction of the elastic portion is increased.

(4) The actuator device recited in (3) above, in which

the first system includes a supporting portion configured to support an acting portion that acts by a reciprocating motion in the predetermined direction.

(5) The actuator device recited in (4) above, in which

the second system includes a sliding portion connected to the supporting portion via the elastic portion.

(6) The actuator device recited in (5) above, in which

the sliding portion has one surface that is oriented in a direction parallel to the predetermined direction and connected to the elastic portion, has the other surface connected to the second magnetic body portion, and is relatively movable in the direction parallel to the predetermined direction by being driving of the driving unit.

(7) The actuator device recited in (6) above, in which

the supporting portion has a hollow structure, and

the sliding portion is housed inside the hollow structure and is relatively movable in the direction parallel to the predetermined direction.

(8) The actuator device recited in any one of (1) to (7) above, in which

the driving unit includes a dielectric elastomer.

(9) The actuator device recited in any one of (1) to (8), in which

in a state where the first system is positioned closest to the magnetic body portion, attraction force by magnetic force of the first magnetic body portion and magnetic force of the second magnetic body portion is larger than restoring force of the elastic portion.

(10) The actuator device recited in any one of (2) to (9) above, in which

in a case where the second system separates the first system from the first magnetic body portion, the driving unit generates driving force in the opposite direction of the predetermined direction, the driving force being larger than a difference between attraction force by magnetic force of the first magnetic body portion and restoring force of the elastic portion.

(11) The actuator device recited in (4) above, further including

a gripping portion that is opened or closed by the reciprocating motion of the acting portion in the predetermined direction.

(12) An end effector including:

a gripping portion; and an actuator unit that generates traction force to the gripping portion, in which

the actuator unit includes

a first magnetic body portion,

a first system movable in a predetermined direction or an opposite direction of the predetermined direction,

a second system including a second magnetic body portion that moves the first system in the predetermined direction by magnetic force generated between the second magnetic body portion and the first magnetic body portion, and a pressurizing portion capable of applying, to the first system, force in the opposite direction of the predetermined direction, and

a driving unit capable of applying, to the second system, force in the predetermined direction or the opposite direction by driving.

(13) The end effector recite in (12) above, in which

the first system includes a supporting portion configured to support an acting portion that causes force in the predetermined direction to act on a gripping portion, and a magnetic body portion that sucks, by magnetic force, the supporting portion in the predetermined direction, and

the second system includes the sliding portion connected to the supporting portion via an elastic portion, and a driving unit that drives the sliding portion in a direction parallel to the predetermined direction.

(14) The end effector recited in (12) or (13) above, in which

the gripping portion converts the traction force in a linear movement direction into gripping force.

(15) The end effector recited in any one of (12) to (14), in which

the gripping portion includes a pair of surgical forceps or another surgical tool.

(16) A surgical system including:

an end effector;

an actuator unit that generates traction force to the end effector; and

a force sensor arranged closer to a proximal end side than the actuator unit.

(17) A surgical system including:

an end effector; and an actuator unit that generates traction force to the end effector,

in which the actuator unit includes

a first system that is sucked by magnetic force of a magnetic body portion and moves, in a predetermined direction, an acting portion that causes the traction force to act on the gripping portion, and

a second system that applies, to the first system, force in an opposite direction of the predetermined direction, and separates the first system from the magnetic body portion.

(18) A surgical robot recited in (17) above, in which

the first system includes a supporting portion configured to support the acting portion that causes force in the predetermined direction to act on a gripping portion, and a magnetic body portion that sucks, by magnetic force, the supporting portion in the predetermined direction, and

the second system includes the sliding portion connected to the supporting portion via an elastic portion, and a driving unit that drives the sliding portion in a direction parallel to the predetermined direction.

(19) The surgical system recited in any one of (16) to (18) above, further including

a force sensor arranged closer to a proximal end side than the actuator unit.

(20) The surgical system recited in (15) or (19), in which

the force sensor includes a strain detection element that detects strain of a strain element and includes an FBG sensor.

### REFERENCE SIGNS LIST

100 Surgical robot
101 Gripping portion
101a, 101b Blade
102 Actuator unit
103 Force sensor
104 Bend portion
301 Acting portion
302 Supporting portion
303 Sliding portion
304 Elastic portion
305 Driving unit (DEA)
306 Magnetic body portion
307 Second magnetic body portion
310 Housing
311 Wire
1001 Strain element
1011 to 1014 Strain detection element (FBG sensor)
1301 to 1304 Optical fiber
1401 Detection unit
1402 Signal processing unit
1403 Difference mode unit
1404 Translational force/moment deriving unit

1. An actuator device comprising:
a first magnetic body portion;
a first system movable in a predetermined direction or an opposite direction of the predetermined direction;
a second system including a second magnetic body portion that moves the first system in the predetermined direction by magnetic force generated between the second magnetic body and the first magnetic body portion, and a pressurizing portion capable of applying, to the first system, force in the opposite direction of the predetermined direction; and
a driving unit capable of applying, to the second system, force in the predetermined direction or the opposite direction by driving.

2. The actuator device according to claim 1, wherein
the pressurizing portion includes an elastic portion.

3. The actuator device according to claim 2, wherein
the more the first system is drawn in the predetermined direction, the more the force in the opposite direction of the elastic portion is increased.

4. The actuator device according to claim 3, wherein
the first system includes a supporting portion configured to support an acting portion that acts by a reciprocating motion in the predetermined direction.

5. The actuator device according to claim 4, wherein
the second system includes a sliding portion connected to the supporting portion via the elastic portion.

6. The actuator device according to claim 5, wherein
the sliding portion has one surface that is oriented in a direction parallel to the predetermined direction and connected to the elastic portion, has the other surface connected to the second magnetic body portion, and is relatively movable in the direction parallel to the predetermined direction by the driving of the driving unit.

7. The actuator device according to claim 6, wherein
the supporting portion has a hollow structure, and
the sliding portion is housed inside the hollow structure and is relatively movable in the direction parallel to the predetermined direction.

8. The actuator device according to claim 1, wherein
the driving unit includes a dielectric elastomer (DEA).

9. The actuator device according to claim 3, wherein
in a state where the first system is positioned closest to the magnetic body portion, attraction force by magnetic force of the first magnetic body portion and magnetic force of the second magnetic body portion is larger than restoring force of the elastic portion.

10. The actuator device according to claim 2, wherein
in a case where the second system separates the first system from the first magnetic body portion, the driving unit generates driving force in the opposite direction of the predetermined direction, the driving force being larger than a difference between attraction force by magnetic force of the first magnetic body portion and restoring force of the elastic portion.

11. The actuator device according to claim 4, further comprising
a gripping portion that is opened or closed by the reciprocating motion of the acting portion in the predetermined direction.

12. An end effector comprising:
a gripping portion; and an actuator unit configured to generate traction force to the gripping portion,
wherein the actuator unit includes
a first magnetic body portion,
a first system movable in a predetermined direction or an opposite direction of the predetermined direction,
a second system including a second magnetic body portion that moves the first system in the predetermined direction by magnetic force generated between the second magnetic body portion and the first magnetic body portion, and a pressurizing portion capable of applying, to the first system, force in the opposite direction of the predetermined direction, and
a driving unit capable of applying, to the second system, force in the predetermined direction or the opposite direction by driving.

13. The end effector according to claim 12, wherein
the first system includes a supporting portion configured to support an acting portion that causes force in the predetermined direction to act on a gripping portion, and a magnetic body portion that sucks, by magnetic force, the supporting portion in the predetermined direction, and
the second system includes the sliding portion connected to the supporting portion via an elastic portion, and a driving unit that drives the sliding portion in a direction parallel to the predetermined direction.

14. The end effector according to claim 12, wherein
the gripping portion converts the traction force in a linear movement direction into gripping force.

15. The end effector according to claim 12, wherein
the gripping portion includes a pair of surgical forceps or another surgical tool.

16. A surgical system comprising:
an end effector;
an actuator unit configured to generate traction force to the end effector; and
a force sensor arranged closer to a proximal end side than the actuator unit.

17. A surgical system comprising:
an end effector; and an actuator unit configured to generate traction force to the end effector,
wherein the actuator unit includes
a first system that is sucked by magnetic force of a magnetic body portion and moves, in a predetermined direction, an acting portion that causes the traction force to act on the gripping portion, and
a second system configured to apply, to the first system, force in an opposite direction of the predetermined direction, and separates the first system from the magnetic body portion.

18. The surgical system according to claim 17, wherein
the first system includes a supporting portion configured to support an acting portion that causes force in the predetermined direction to act on a gripping portion, and a magnetic body portion configured to suck, by magnetic force, the supporting portion in the predetermined direction, and
the second system includes the sliding portion connected to the supporting portion via an elastic portion, and a driving unit configured to drive the sliding portion in a direction parallel to the predetermined direction.

19. The surgical system according to claim 17, further comprising
a force sensor arranged closer to a proximal end side than the actuator unit.

20. The surgical system according to claim 16, wherein
the force sensor includes a strain detection element configured to detect strain of a strain element and including a fiber Bragg grating (FBG) sensor.
